(19) United States
(12) Patent Application Publication
(10) Pub. No.: US 2023/0383339 A1
(43) Pub. Date: Nov. 30, 2023

(54) MULTIPLEXED METHYLATED DNA IMMUNOPRECIPITATION SEQUENCING TO STUDY DNA METHYLATION USING LOW AMOUNTS OF DNA

(71) Applicant: ARIZONA BOARD OF REGENTS ON BEHALF OF ARIZONA STATE UNIVERSITY, Scottsdale, AZ (US)

(72) Inventors: Joshua LABAER, Chandler, AZ (US); Vel MURUGAN, Chandler, AZ (US); Inam RIDHA, Tempe, AZ (US)

(21) Appl. No.: 18/248,215

(22) PCT Filed: Oct. 7, 2021
(86) PCT No.: PCT/US2021/053990

§ 371 Date: Apr. 6, 2023

Related U.S. Application Data
(60) Provisional application No. 63/089,967, filed on Oct. 9, 2020.

(51) Int. Cl. C12Q 1/6855; C12N 15/10; C12Q 1/6876

(52) U.S. Cl. CPC C12Q 1/6855; C12N 15/1093; C12Q 1/6876; C12Q 2600/154

(57) ABSTRACT

The disclosure relates to methods for multiplexed detection of DNA methylation in pooled DNA libraries. In particular, provided herein are methods for detection of DNA methylation in pooled DNA libraries using enzymatic digestion by a micrococcal nuclease, methylated DNA immunoprecipitation, and quantitative amplification.

### CROSS-REFERENCE TO RELATED APPLICATIONS

This application claims the benefit of U.S. Application No. 63/089,967 filed on Oct. 9, 2020, which is incorporated herein by reference in its entirety.

### STATEMENT REGARDING FEDERALLY SPONSORED RESEARCH AND

### DEVELOPMENT

This invention was made with government support under W911NF-19-C-0039 awarded by the Army Research Office. The government has certain rights in the invention.

### BACKGROUND

DNA methylation is one of the most widely studied epigenetic marks that play a crucial role in gene silencing, cell fate decisions, and disease development. Methylation of DNA involves covalent modification of the pyrimidine ring of cytosine nucleotide at the C-5 position by the addition of a methyl group and is mostly found in the form of symmetrical CpG dinucleotides in mammalian cells. Several techniques are known in the art to study the variability in methylation patterns and simultaneously transform that information into quantitative and measurable signals. For instance, methods such as whole-genome bisulfite sequencing (WGBS) investigate single base methylation or enrichment of methylated DNA fragments through immunoprecipitations using antibody or methyl binding domain proteins (MBD). WGBS combines sodium bisulfite conversion of input DNA with high-throughput DNA sequencing. Although bisulfite sequencing permits analysis of DNA methylation with single base resolution, bisulfite treatment causes substantial DNA degradation and requires DNA purification. To have sufficient amounts of DNA following treatment, microgram quantities of input DNA are required. Other limitations of WGBS include its high cost, particularly for large sample sizes, and its inability to distinguish between 5-methylcytosine (5mC) and hydroxymethylcytosine (5hmC) as distinct epigenetic modifications.

Methylated DNA immunoprecipitation followed by sequencing (MeDIP-seq) is another widely used method to study DNA methylation profiles. MeDIP enriches methylated DNA fragments using monoclonal antibodies against 5mC, and has been adapted to detect 5-hydroxymethylcytosine (5hmC) DNA. The methylation-enriched sample, prepared by immunoprecipitation, can be analyzed using high-throughput sequencing to identify the methylated region by comparing it to an “input” control that was not subjected to immunoprecipitation. While MeDIP does not provide information with single-base resolution, it has sufficient resolution to detect differentially methylated regions (DMRs), which are functionally more important than single methylation polymorphisms (SMPs). At first MeDIP-seq was required to have microgram of input DNA to start with, however several protocols have been developed to use a smaller amount of input DNA. For instance, Taiwo et al. (Taiwo, O.; Wilson, G. A.; Morris, T.; Seisenberger, S.; Reik, W.; Pearce, D.; Beck, S.; Butcher, L. M., Methylome analysis using MeDIP-seq with low DNA concentrations. Nat Protoc 2012, 7 (4), 617-36) developed a protocol to use 50 ng of DNA, but multiplexed analysis was not possible. Furthermore, the methylated immunoprecipitation protocol required 3-5-days to complete. In another method, PCR amplification was used for a low amount of DNA after immunoprecipitation followed by sequencing. See Zhao, M. T.; Whyte, J. J.; Hopkins, G. M.; Kirk, M. D.; Prather, R. S., Methylated DNA immunoprecipitation and high-throughput sequencing (MeDIP-seq) using low amounts of genomic DNA. Cell Reprogram 2014, 16 (3), 175-84. Fold enrichment with PCR amplification was only possible for highly methylated regions, and few samples could be processed at once. Thus, current protocols for sequencing of methylated DNA are labor intensive, expensive, and incapable of multiplexed analysis. Accordingly, there remains a need in the art for improved methods of DNA immunoprecipitation that provide for faster processing and analysis for aberrant DNA methylation.

### BRIEF DESCRIPTION OF THE DRAWINGS

FIG. 1. Multiplexed Methylated DNA Immunoprecipitation sequencing workflow.

FIG. 2. Stages and component steps of bioinformatics data analysis.

FIGS. 3A-3D. Specificity (A) and fold change ratio (B) for individual and pooled samples. 10 different library DNA went through MeDIP individually. Also, a pool containing all those 10 samples went through IP simultaneously. The plots show a comparison between an individual (n=10) and pooled samples. (C) Empirical GC content in the MeDIP enriched samples (orange line) compared to the theoretical distribution (blue and green line). (D). Representative images of correlation of read coverage between individual and pooled samples (For these studies, we carried out n=10 for each condition).

FIGS. 4A-4B. Specificity (A) and fold change ratio (B) for a pool of equal distribution of starting DNA vs different distribution of starting DNA For these studies, n=3 carried out for each condition.

FIGS. 5A-5B. Specificity (A) and fold change ratio (B) for a pool of DNA carried through multiplexing MeDIP. n=3 experiment was carried out for each condition. The anti-5-methylcytosine antibody was used in each pull-down experiment with different amounts of starting materials (4 ng, 20 ng, 40 ng, 100 ng, 200 ng, 400 ng). Pools contain 4 different DNA samples. Statistical analysis between groups was determined using one-way ANOVA and Tukey test; p-values<0.05 were considered statistically significant. For these studies, n=3 carried out for each condition, and in the figure, *: p-value<0.05, **: p-value<0.01, ***: p-value<0.001.

FIG. 6A-6B. (A) Representative image of agarose gel (1.5%) electrophoresis for DNA fragmented by micrococcal nuclease (MNase) digestion. The chromatin has been isolated from peripheral blood mononuclear cells (PBMCs). The left lane is 100 bp marker. MNase digesting was done to have about 30% mononucleosomes and 25% dinucleosomes (analyzed by ImageJ). (B) Pattern of fragments after library prep. The fragments shifted by 150 bp due to adding the Indexes during library prep. DNA library has proceeded for multiplexing and MeDIP

FIG. 7. Corresponding integrative genome viewer (IGV) tracks of ChIP-seq peaksets at a single representative to compare different histone marks tracks at arbitrary locus.

FIG. 8A-8B. Specificity (A) and fold change ratio (B) for a pool of equal distribution of starting DNA vs. different distribution of starting DNA For these studies, we carried out n=3 for each condition.

### DETAILED DESCRIPTION

Provided herein are methods and compositions for multiplexing of different prepared libraries in methylated DNA immunoprecipitation, where the method involves performing micrococcal nuclease (MNase) digestion and using MNase-digested DNA for multiplexed methylated DNA immunoprecipitation. Advantages of these methods and compositions are multifold and include, without limitation, that minimal amounts of DNA are needed to perform methylated DNA immunoprecipitation (MeDIP). For instance, MeDIP can be performed according to the methods of this disclosure using as low as 10 ng DNA for specificity of >90%, and as low as 1 ng DNA for specificity of at least about 73%. The methods of this disclosure are also cost-effective and time-saving, and substantially reduce time, reagents, error, and complexity of the MeDIP process, which minimizes hands-on time and simplifies the traditional labor-intensiveness of other methods.

DNA methylation is one of the most widely studied epigenetic marks that play a crucial role in gene silencing, cell fate decisions, and disease development. It involves covalent modification of pyrimidine ring of cytosine nucleotide at the C-5 position by the addition of methyl group and it mostly found as symmetrical CpG dinucleotides in mammalian cells. Several techniques can study the variability in methylation patterns and simultaneously transform that information into quantitative and measurable signals. This includes methods to investigate single base methylation such as whole-genome bisulfite sequencing (WGBS), or enrichment of methylated DNA fragments through immunoprecipitations using antibody or methyl binding domain proteins (MBD). WGBS combines sodium bisulfite conversion of input DNA with high-throughput DNA sequencing. Although bisulfate sequencing leads to analyze DNA methylation at single-based resolution, Bisulfite treatment causes substantial DNA degradation. Moreover, to remove the sodium bisulfite, the DNA must be purified. Thus, WGBS requires a microgram amount of input DNA, which may be limiting in certain contexts and in certain experimental systems. Gu, H.; Smith, Z. D.; Bock, C.; Boyle, P.; Gnirke, A.; Meissner, A., Preparation of reduced representation bisulfite sequencing libraries for genome-scale DNA methylation profiling. Nat Protoc 2011, 6 (4), 468-81. Other limitations of WGBS are the high cost of the technology, especially on large sample sizes, and the inability of this method to distinguish between 5-methylcytosine (5 mC) and hydroxy-methylcytosine (5 hmC), which constitute distinct epigenetic modifications.

Methylated DNA immunoprecipitation followed by sequencing (MeDIP-seq) is another widely used method to study DNA methylation profiles. MeDIP enriches methylated DNA fragments using monoclonal antibodies against 5-methyl cytosine. The enriched immunoprecipitated sample is then analyzed by high-throughput sequencing to identify the methylated regions by comparing it to non-enriched, input DNA that has not been subjected to immunoprecipitation. This technique was also adapted to detect 5-hydroxymethylcytosine (5hmC) DNA, using an antibody directed to 5-hydroxymethylcytosine. See, for example, Rauluseviciute, I.; Drablos, F.; Rye, M. B., DNA methylation data by sequencing: experimental approaches and recommendations for tools and pipelines for data analysis. Clin. Epigenetics 2019, 11 (1), 193. Although MeDIP does not provide information at single-base resolution, it can provide sufficient resolution to study differentially methylated regions (DMRs), which are functionally more important than single methylation polymorphisms (SMPs). See, for example, Wardenaar, R.; Liu, H.; Colot, V.; Colome-Tatche, M.; Johannes, F., Evaluation of MeDIP-chip in the context of whole-genome bisulfite sequencing (WGBS-seq) in Arabidopsis. Methods Mol Biol 2013, 1067, 203-24; Schmitz, R. J.; Schultz, M. D.; Lewsey, M. G.; O′Malley, R. C.; Urich, M. A.; Libiger, O.; Schork, N. J.; Ecker, J. R., Transgenerational epigenetic instability is a source of novel methylation variants. Science 2011, 334 (6054), 369-73; and Becker, C.; Hagmann, J.; Muller, J.; Koenig, D.; Stegle, O.; Borgwardt, K.; Weigel, D., Spontaneous epigenetic variation in the Arabidopsis thaliana methylome. Nature 2011, 480 (7376), 245-9. Initial protocols for MeDIP-seq required a microgram of sample input DNA, however there are several protocols developed to use a smaller amount of input DNA. Taiwo et al. developed a protocol to use as little as 50 ng of DNA. However, this method requires 3-5 days of processing for each sample. See Taiwo et al., supra. In another method, PCR amplification was used for a low amount of DNA after immunoprecipitation followed by sequencing. See Zhao, et al., supra. Significant enrichment was observed for only a highly methylated region in their study. In addition, the number of samples that could be processed at one time was also limited in all above-described protocols.

Accordingly, in a first aspect provided herein, is a method for multiplexed detection of DNA methylation in pooled DNA samples. In particular, provided herein is a method for detection of DNA methylation in pooled DNA samples using enzymatic digestion by a micrococcal nuclease, methylated DNA immunoprecipitation, and quantitative amplification. As demonstrated herein, the method of the current disclosure allows analysis of large numbers of DNA molecules, including from multiple genomic DNA sources, in a parallel manner while using a lower amount of input DNA than is required for conventional techniques.

In a first step, the method comprises contacting a micrococcal nuclease (MNase) to two or more DNA samples. Subsequently, the DNA fragments in each sample are ligated with a sample-specific adapter to generate an adapter ligated DNA library from each sample. Sample-specific adapter ligation facilitates pooling of two or more libraries. Next, two or more libraries are combined and methylated DNA is enriched by immunoprecipitation. Finally, the DNA is sequenced. The sample identity of each enriched adapter ligated DNA sequence is determined during the analysis of the sequence based upon the unique adapter that is ligated to the DNA fragments during library construction. In some cases, the methods of this disclosure use DNA amounts as low as 10 ng for greater than (>) 95% specificity, and DNA amounts as low as 1 ng for >70% specificity. Every run of multiplexed MeDIP sequencing can contain numerous different samples, which means the methods of this disclosure are particularly well suited for simultaneous analysis of a large number of samples. In some cases, the pooled DNA sample comprises double-stranded DNA. In some cases, the double-stranded DNA is genomic DNA or cDNA obtained from multiple sources (e.g., from 2 to 15 individual sources). In some embodiments, the multiple sources are from more than 15 individual sources. In some embodiments, the multiple sources are from 2 to 100 sources.

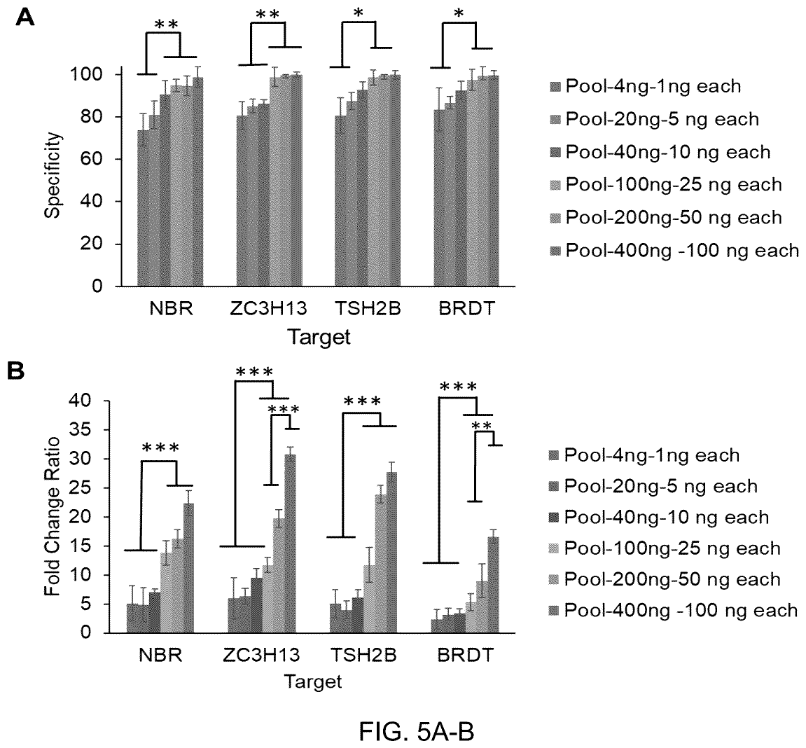

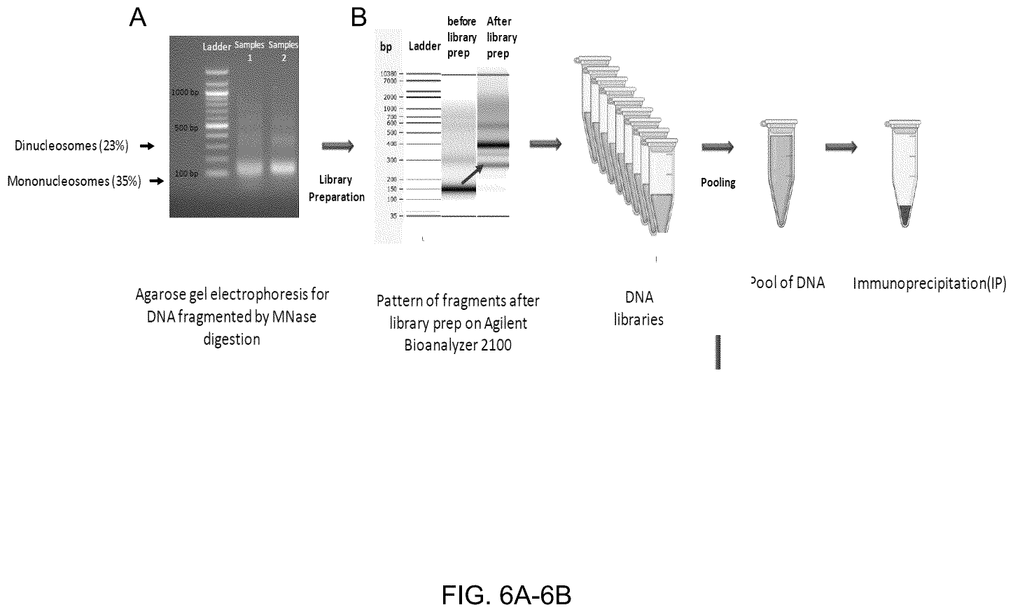

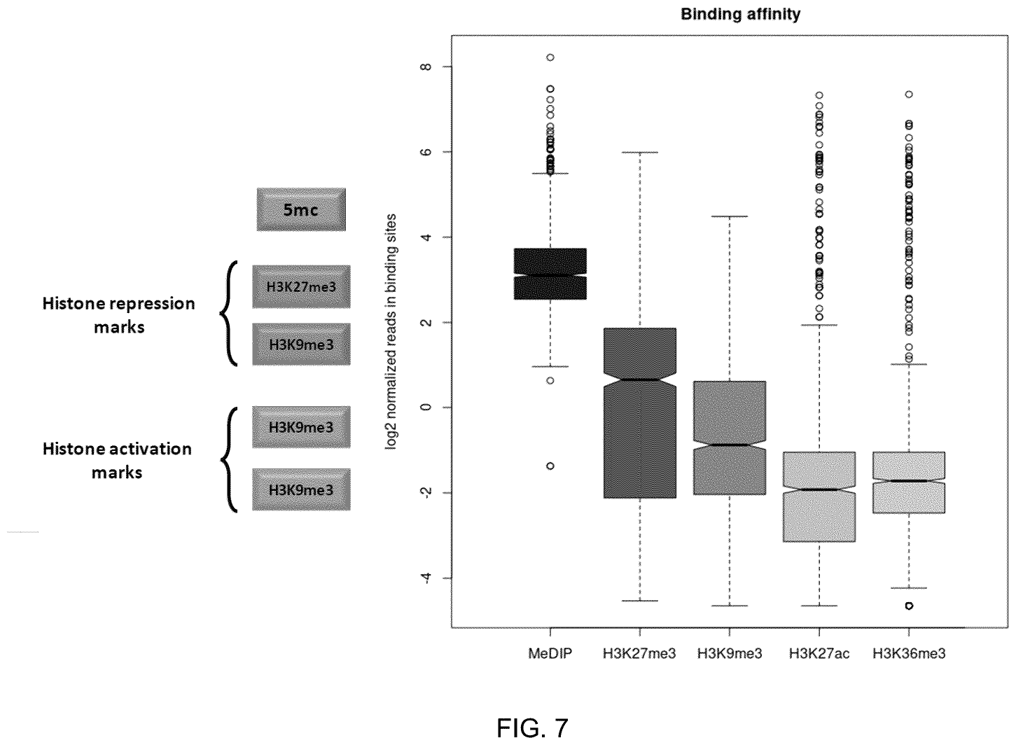

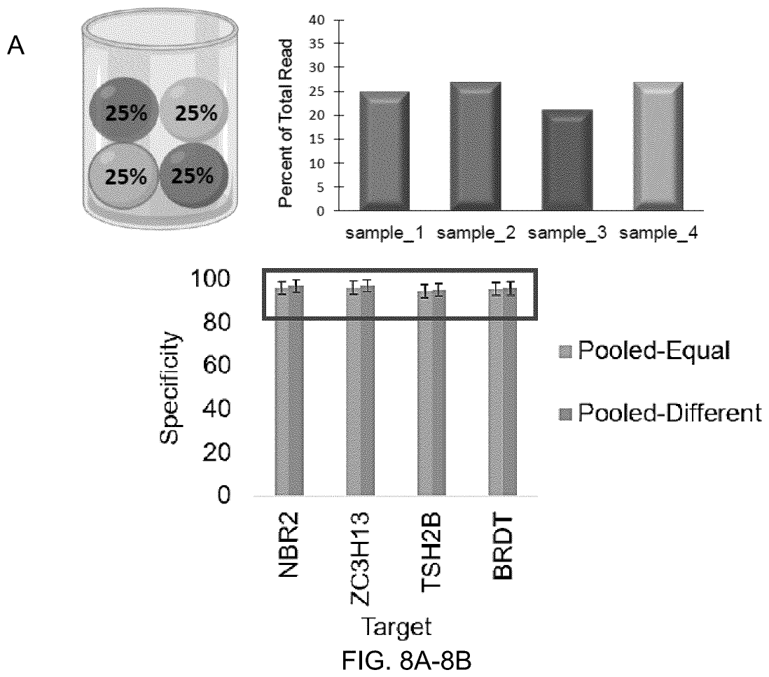

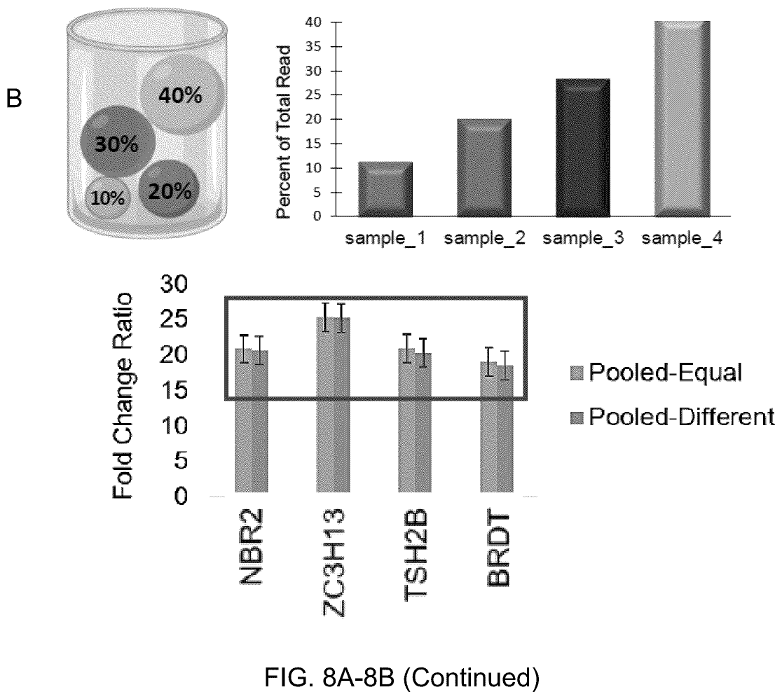

Multiplexed MeDIP has the advantages of being cost-effective and timesaving. In addition, it also requires a small amount of starting DNA. For the experiment presented in FIGS. 5A-5B, varying amounts of each DNA library (4 ng, 20 ng, 40 ng, 100 ng, 200 ng, 400 ng from each source) were tested in triplicate. Each pooled library contained genomic DNA from 4 different sources. For each pooled library, DNA from four separate sources were independently ligated to unique indexes and pooled. The pooled library was then precipitated using a 5-methylcytosine-specific antibody. The precipitated DNA was analyzed using quantitative polymerase chain reaction (qPCR) to determine the specificity and fold enrichment ratios. Fold change was calculated using Equation 1.

Fold Change=2^{Input Ct-MeDIP Ct} Equation 1

In Equation 1, cycle threshold (Ct) is the cycle at which the amplification of the indicated sample crosses an arbitrary threshold that is common between all samples in the assay, or in some exemplary embodiments, amplification becomes detectable.

$$Fold Change Ratio = \frac{Fold Change Methylated}{Fold Change Unmethylated} \qquad Equation 2$$

$$Percent Recovery = \frac{Amount of Enriched sample DNA from IP}{\begin{matrix}Amount of Enriched Input DNA \\ from input \times \frac{s a m p l e}{i n p u t}\end{matrix}} \times 100 \qquad Equation 3$$

In Equation 3, Enriched sample DNA from IP and Enriched Input DNA from input are measured in nanograms (ng).

$$Specificity = 1 - \frac{P e rcent Recovery Unmethylated}{P e rcent Recovery Methylated} \qquad Equation 4$$

By increasing the starting material, the amount of overall DNA captured was increased. FIGS. 5A-5B show the specificity and fold enrichment plots for each pool. As FIG. 5A indicates, using as little as 10 ng of DNA per sample results in acceptable specificity (>90%). However, there is a significant drop in sensitivity when using between 1 ng and 5 ng of DNA per library, resulting in 73 to 85% of specificity. Lowering the DNA amount to 1 ng reflects the limitation in distinguishing methylated and unmethylated DNA; however, despite the loss of specificity when using 1 ng of starting material, the assay is still more than 70% specific for methylated regions. FIG. 5B illustrates the fold enrichment ratio achieved for starting the DNA amount as little as 1 ng in the pool. Using as little as 10 ng of DNA would result in acceptable specificity and fold enrichment ratio. However, there is a significant reduction in fold enrichment ratio of 1 ng and 5 ng of DNA compared to 50 ng and 100 ng of starting DNA. Moreover, there was no significant difference in specificity and fold enrichment ratio using 50 ng and 100 ng of starting DNA. The reduction of starting materials to 25 ng also results in comparable fold enrichment ratio and specificity vs individuals or 50 ng of DNA. Similarly, the two experiments indicated that using as low as 5 ng and 1 ng would result in a significantly lower but still detectable fold enrichment ratio. While it is not recommended to use less than 10 ng individual DNA samples, high specificity and fold enrichment is obtainable if at least 10 ng of each individual DNA sample is used in the pooled sample.

In some embodiments, the pooled DNA sample comprises about 5 ng to about 100 ng or more (e.g., about 5, 10, 15, 20, 25, 30, 35, 40, 45, 50, 55, 60, 65, 70, 75, 80, 85, 90, 95, 100 ng or more) of genomic DNA of each individual source. Preferably, the pooled DNA sample comprises at least 10 ng genomic DNA of each individual source. As demonstrated herein, this amount yields results with greater than 90% specificity, although it will be understood that smaller amounts of each sample may be used if a lower percentage of specificity is acceptable. In some cases, the pooled DNA sample comprises less than 100 ng genomic DNA of each individual source.

In some embodiments, disclosed herein are kits. In some embodiments, the kits comprise (a) micrococcal nuclease; and (b) an antibody specific for methylated DNA. In some embodiments, the kits comprise sample-specific adapters and reagents for ligating the adapters to nucleosome-protected fragments. In some embodiments, the kits comprise primers that hybridize with the sample-specific adapters. In some embodiments, the kits comprise reagents for amplifying the nucleosome-protected fragments. In some embodiments, the reagents for amplifying comprise reagents for polymerase chain reaction (PCR). In some embodiments, the reagents for PCR comprise a high-fidelity polymerase. In some embodiments, the kits comprise reagents for next generation sequencing.

As used herein, the term “micrococcal nuclease” or “MNase” refers to a Ca²⁺ dependent endo-and exo-nuclease that preferentially digests DNA not bound to nucleosomes, releases nucleosomes from chromatin, and enriches for nucleosome-protected DNA fragments. In some embodiments, MNase is contacted to the sample in an amount sufficient to digest chromatin in linker regions of the DNA between nucleosomes. In some embodiments, MNase is contacted to the sample at about 40° to 70° C., inclusive. In some embodiments, the MNase is contacted to the sample at about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70° C. In some embodiments, the MNase is contacted to the sample resulting in about 30% mono-nucleosomal DNA and about 25% di-nucleosomal DNA. As used herein, “mono-nucleosomal DNA” refers to a DNA molecule that forms a single nucleosome. As used herein, “di-nucleosomal DNA” refers to a DNA molecule that forms two nucleosomes. As a result of contacting MNase to the sample, chromatin comprising DNA wound around histones is enzymatically digested to form single nucleosomes and nucleosome-protected DNA fragments.

The MNase reaction can be stopped via the addition of one or more of EGTA, EDTA, and a serine protease such as Proteinase K, which enzymatically digests all of the proteins including histones. The resulting DNA can be purified using any appropriate techniques including, for example, purification by contacting the DNA to a solid support comprising one or more nucleic acid binding reagents (e.g., a DNA purification column, beads).

Following MNase treatment, protein digestion, and DNA purification, a sample comprising nucleosome-protected DNA fragments is obtained. Subsequently, sample-specific adaptors are ligated to the nucleosome-protected DNA fragments to create a library of adaptor-ligated DNA fragments.

Next, the adaptor-ligated DNA fragments can be pooled to form a pooled DNA library. As used herein, the term “library” refers to a plurality of nucleic acids derived from a single source. As used herein, the term “pooled library” refers to the product of combining multiple libraries, each from a different source. In some cases, a “library” comprises a plurality (e.g., collection) of “library fragments” which are nucleic acids produced by fragmenting a larger nucleic acid, e.g., physical (e.g., shearing), enzymatic (e.g., by nuclease), chemical treatment, and/or amplification (e.g., PCR). In some embodiments, a library preparation is performed before enrichment by methylated DNA immunoprecipitation.

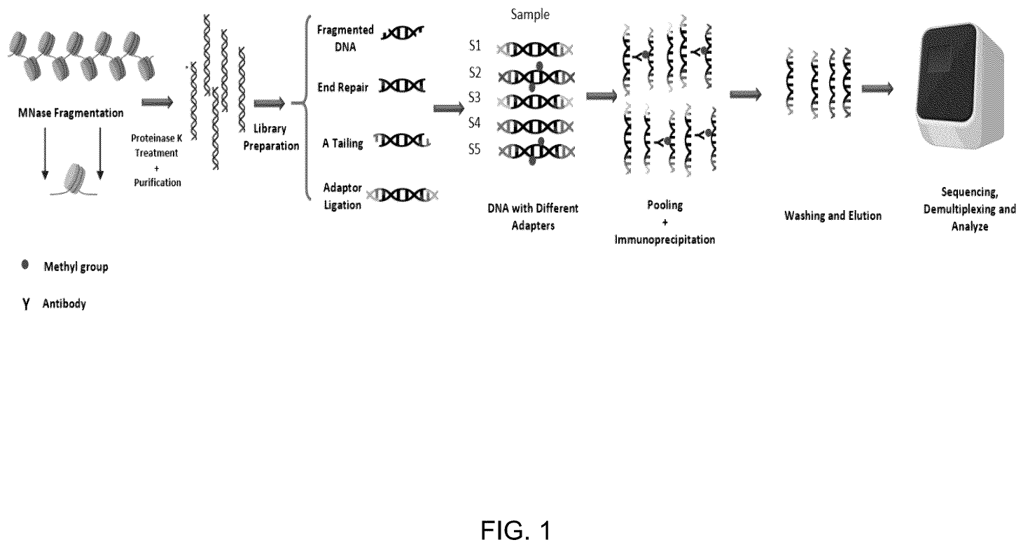

As illustrated in FIG. 1, in some embodiments, library preparation comprises ligating an adapter to the purified nucleosome-protected DNA fragments prior to pooling. Thus, each individual DNA library comprises a unique adapter sequence that can be used to bioinformatically separate DNA sequences in sequencing data derived from pooled DNA libraries. The adapter can be any type of adapter known in the art including, but not limited to, a conventional duplex or double stranded adapter. The adapter can further comprise a known or universal sequence, thus allowing generation and/or use of sequence specific primers for the known or universal sequence. In some cases, an adapter comprises a barcode. In some cases, unique adapters are used for each genomic DNA source. DNA library preparation can also comprise subjecting the nucleosome-protected DNA fragments to end repair which can include, without limitation, generation of blunt ends, non-blunt ends (i.e., sticky or cohesive ends), or single base overhangs such as the addition of a single dA nucleotide to the 3′-end of the double-stranded DNA fragment using a polymerase lacking 3′-exonuclease activity. End repair can be performed using any number of enzymes and/or methods known in the art, and typically is performed prior to the addition of adaptors.

Methylated DNA immunoprecipitation is then performed on the pooled DNA library, thereby producing a multiplexed methylated enriched DNA sample.

The multiplexed methylated enriched DNA sample is then amplified. Numerous amplification techniques are known in the art including, without limitation, quantitative PCR (qPCR), quantitative fluorescent PCR (QF-PCR), multiplex fluorescent PCR (MF-PCR), real time PCR (RT-PCR), nested PCR, and isothermal amplification techniques. Preferably, amplifying comprises quantitative polymerase chain reaction (qPCR).

Next, the amplified sample is sequenced to detect DNA methylation. Any appropriate means of DNA sequencing can be used. For example, Sanger sequencing or next generation sequencing (NGS) can be used. As used herein, the term “next generation sequence” refers to higher throughput and/or lower cost nucleic acid sequencing technologies. Because the libraries are subjected to immunoprecipitation with an antibody that recognizes methylated DNA, the pooled libraries are highly enriched for sequences that have been methylated. In other words, the enrichment of the samples by immunoprecipitation for methylated DNA insures that the DNA being sequenced is methylated, as the sequencing of the DNA fragments themselves cannot distinguish methylated from non-methylated DNA.

As used herein, “next generation sequencing reagents” or “NGS reagents” refers to reagents that are used in the process of preparing samples for and performing next generation sequencing (NGS). By way of example, but not by way of limitation, in some embodiments, NGS reagents comprise reagents for library preparation, clonal amplification, sequencing by addition, and for other processes generally related to NGS that are well known in the art including appropriate buffers and washing solutions.

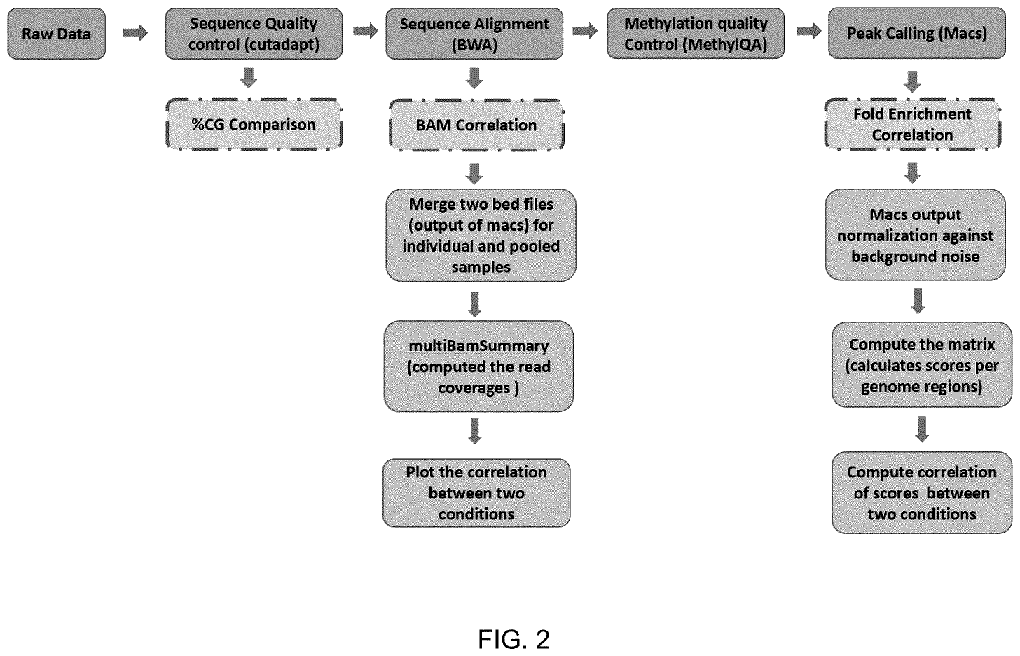

Any appropriate method of analyzing sequence results obtained according to the methods of this disclosure can be used. In some cases, a software program can be used to remove adapter sequences from raw sequencing reads. As illustrated in FIG. 2, cleaned reads (i.e., having adapter sequences removed) can be aligned to identify enriched areas of DNA methylation in the genome. In some cases, sequencing results from pooled samples can be compared to results obtained from individual samples or between pooled samples. The read coverage (number of unique reads mapped at a given nucleotide) over a large number of regions can be determined for each of the inputted sequence files (e.g., mapped sequencing reads, control read). The sequencing results can be analyzed, for example, for fold enrichment of CpG islands.

In another aspect, provided herein is a method for detecting aberrant methylation in target gene(s) in a DNA sample obtained from a subject, where the aberrant methylation of the target gene(s) is associated with various diseases. Accordingly, the methods of this disclosure are particularly well suited for detection and analysis of aberrant methylation of genes associated with diseases such as, for example, colorectal cancer, Prader-Willi, Angelman, Beckwith-Wiedmann syndromes, and can be used for clinical diagnostics. In particular, the methods of this disclosure can be performed to detect clusters of CG dinucleotides called CpG islands. As used herein, the term “CpG island” refers to DNA sequences, typically more than 200 base pairs long, having CG content greater than 50% and an observed/expected CpG ratio of more than 60%. Methylation of CpG islands is typically associated with gene silencing, while demethylation of these sites enables transcription. Preferably, the method is performed to analyze a specific locus, in which case the investigated region is preferably unmethylated in normal tissue and methylated in cancerous tissue or vice versa. Preferably, the methylation levels should enable differentiation between the two statuses of the samples (e.g., test sample vs. control).

In some cases, the method is performed to analyze the methylation state of samples at one or more of the following genetic loci: Bromodomain Testis-Specific Factor (BRDT), Neighbor of BRCA1 gene 2 (NBR2), Zinc Finger CCCH Domain-Containing Protein 13 (ZC3H13), and testis-specific variant of H2B (TSH2B). TSH2B and BRDT genes are transcribed exclusively in testis, and CpG sites of these genes are methylated in all somatic tissue. The ZC3H13 gene encodes one of three components of a complex that mediates N6-methyladenosine (m⁶A) methylation, which is the most abundant mRNA modification in eukaryotes. The NBR2 locus is located within a large CpG island and has been shown to be methylated in most somatic cells.

In some cases, the method comprises obtaining a sample comprising double-stranded DNA from a subject. In some cases, the sample is from a subject (e.g., human subject) who is healthy. In other cases, the sample is from a subject affected by a genetic disease, a carrier for a genetic disease, or at risk for developing or passing down a genetic disease, where a genetic disease is any disease that can be linked to genetic variation such as aberrant methylation but also mutations, insertions, additions, deletions, translocation, point mutation, trinucleotide repeat disorders, and/or single nucleotide polymorphisms (SNPs).

The terms “library” and “sequencing library” are used herein to refer to a pool of DNA fragments with adapters attached. Adapters are commonly designed to interact with a specific sequencing platform, e.g., the surface of a flow-cell (Illumina) or beads (Ion Torrent), to facilitate a sequencing reaction.

To track the source of each DNA fragment in a pooled sample, a unique molecular barcode (or combination of multiple barcodes) is included in the adapters that are ligated to the DNA fragments in each library. During the sequencing reaction, the sequencer reads this barcode sequence in addition to the DNA's biological base sequence. The barcodes are then used to assign each DNA to its sample of origin during data analysis, a process termed “demultiplexing”.

The indexing strategy used for a sequencing reaction should be selected based on the number of pooled samples and the level of accuracy desired. For example, unique dual indexing, in which unique identifiers are added to both ends of the DNA fragments, may be used to ensure that libraries will demultiplex with high accuracy. Adapters may also include unique molecular identifiers (UMIs), short sequences, often with degenerate bases, that incorporate a unique barcode onto each molecule within a given sample library. UMIs reduce the rate of false-positive variant calls and increase sensitivity of variant detection by allowing true variants to be distinguished from errors introduced during library preparation, target enrichment, or sequencing. Many index sequences and adapter sets are commercially available including, for example, SeqCap Dual End Adapters from Roche, xGen Dual Index UMI Adapters from IDT, and TruSeq UD Indexes from Illumina.

As used herein, “barcode” refers to a nucleotide sequence of any length that is used to identify, for example, nucleotide sequences that are derived from a single sample. An exemplary property of a barcode is the ability to distinguish the sequence of the barcode from any known sequence present in the sample, thereby rendering the barcode sequence informatically distinct and permitting identification or quantification of any nucleotide sequence comprising the barcode. In some embodiments, a barcode may be 6-8 nucleotides in length. Each barcode must be detected in a single sequencing “read.” Therefore, barcode length is, in principle, dictated by the sequencing platform used to analyze the samples.

As used herein, “primer” refers to a single-stranded nucleotide. In some embodiments, a primer is used to initiate semi-conservative replication of nucleic acids.

As used herein, “pooling” refers to the combination of multiple libraries, each with a unique barcode, to be sequenced in a single run. The sequencer to be used and the desired sequencing depth should dictate the number of samples that are pooled. For example, for some applications it is advantageous to pool fewer than 12 libraries to achieve greater sequencing depth, whereas for other applications it may be advisable to pool more than 100 libraries.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26 (3/4):227-259; and Owczarzy et al., 2008, Biochemistry, 47: 5336-5353, which are incorporated herein by reference).

As used herein, “amplification” refers to the process of semi-conservatively replicating nucleic acid strands by enzyme-catalyzed extension. Exemplary enzymes for amplification of nucleic acids in the current disclosure include, for example, nucleic acid polymerases. In some embodiments, amplification is carried out with a high fidelity polymerase, such as Q5, with the technique known as the polymerase chain reaction (PCR). Amplification can be performed with natural and non-natural nucleotide bases, ribonucleotide bases or deoxyribonucleotide bases, labeled nucleotide bases, and the like.

As used herein, “ligation” refers to the joining of two nucleic acid molecules through the formation of covalent phosphodiester bonds. Ligation may involve the joining of double-stranded or single stranded nucleic acid molecules. In some embodiments, two blunt-ended nucleic acid duplexes are ligated together. In some embodiments, two nucleic acid duplexes that have single-stranded regions that are substantially complementary to one another allowing hybridization of the two nucleic acid duplexes are ligated to one another.

As used herein, “nucleosome protected” refers to DNA that is wound around histones such that the DNA is less accessible to degradation by, for example, micrococcal nuclease. In some embodiments, nucleosome protected DNA is freed from nucleosome protection by contacting a solution comprising nucleosome protected DNA with a serine protease, for example, proteinase K, which degrades the histones and frees the DNA.

As used herein, “immunoprecipitation” refers to the process of selectively precipitating a target of interest using an antibody specific for said target. In some embodiments, immunoprecipitation can selectively precipitate, and enrich, one target from a solution containing many targets. In some embodiments, an antibody specific for 5-methycytosine is used to precipitate DNA with that particular modification. In some embodiments, an antibody specific for 5-hydroxymethylcytosine is used to precipitate DNA with that particular modification. Antibodies used for immunoprecipitation are often bound to a solid support. In some embodiments, the solid support comprises beads. Antibodies specific for 5-methylcytosine and 5-hydroxymethylcytosine for use in the methods of the current disclosure are well known in the art and commercially available. The present technology is not intended to be limited by a particular antibody.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids and/or other constructs of the invention may be isolated. As used herein, “isolated” means to separate from at least some of the components with which it is usually associated whether it is derived from a naturally occurring source or made synthetically, in whole or in part.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Various exemplary embodiments of methods according to this disclosure, in addition to those shown and described herein. Those skilled in the art will recognize, or be able to ascertain using no more than routine experimentation, many equivalents to the specific embodiments of the invention described herein. Such equivalents are intended to be encompassed by the claims.

### EXAMPLES

### Micrococcal Nuclease Digestion (Native IP)

The Micrococcal Nuclease Digestion method was designed based on previous work but with adaptations. PBMCs were centrifuged twice at 600×g for 10 minutes at 20° C. and washed with RPMI+ 20% FBS each time. Then, the cell pellet was resuspended in PBS buffer at 7,500 cells per 1 μL concentration. An equal volume of the MNase digestion buffer (0.02 U/μL MNase (Thermo Scientific, Cat. No: 88216) in 2× lysis buffer (Table 1) was added to the cell suspension and gently vortexed to mix. The cell suspension then was incubated on ice for 20 minutes followed by incubation at 55° C. water bath for 10 minutes. To inactivate the digestion reaction EGTA (30 mM final concentration) was used. Then the digested chromatin was treated with proteinase k (Ambion™) at 55° C. and then pre-cleared with of 1:2 KAPA Pure beads (Roche Cat. No: 07983298001). The quality and quantity of fragmented DNA were determined using agarose gel (1.5%) electrophoresis and Qubit fluorometer quantification. The resulting purified DNA was used for library construction.

TABLE 1
2x Lysis buffer stock preparation for MNase digestion

| | Volume |
|---|---|
| Reagents | (mL) |
| 100 mM Tris-HCl (1M, pH 8.0) | 20 |
| 300 mM NaCl (5M) | 12 |
| 2% Triton X-100 (25%) | 16 |
| 0.2% sodium deoxycholate | 3.2 |
| (DOC, 12.5%) | |
| 10 mM CaCl2 (1M) | 2 |
| 10 mM sodium butyrate (800 mM) | 2.5 |
| H2O | 144.3 |
| 1 U/μL MNase Stock solution | 4 |
| Total Volume | 200 |

### Library Preparation

Illumina KAPA LTP Library Preparation Kit was adapted for library construction (Cat. No: KK8233).

### Methylated DNA Immunoprecipitation Sequencing (MeDIP-Seq)

FIG. 1 illustrates an exemplary workflow for multiplexing MeDIP. As illustrated, the method involves immunocapturing of methylated DNA fragments using an antibody specific for methylated cytosines. Affinity of the antibody used in MeDIP for methylated cytosines enables the detection of any methylated cytosine and is not restricted to the analysis of CpG island. MeDIP was performed using a MeDIP kit according to the manufacturer's instructions (Active Motif, catalog number 55009). The procedure for immunoprecipitating methylated genomic DNA fragments was modified to use different amounts of input DNA and required antibody. Specifically, low amounts of fragmented DNA (50 ng, 100 ng, 200 ng, 500 ng, 1 μg) were used with different amounts of antibody (0.5 μg to 8 μg), and the enrichment reactions were carried out in low binding tubes (Eppendorf LoBind tubes) to minimize DNA loss during the MeDIP procedure. Library size selection was performed on the enriched material, and the size-selected enriched fraction of the immunoprecipitated (IP) sample DNA was amplified followed by a real-time quantitative polymerase-chain-reaction (qPCR) technique to analyze the final enrichment for methylated DNA. The amplified material was sequenced. To confirm the enrichment of methylated DNA in the IP samples, four primers set specific for the highly methylated region as positive controls, and one primer for the unmethylated region was used as a negative control (Table 2). Specificity was calculated based on percent recovery.

TABLE 2
Primers used in qPCR to assess the enrichment of pooled down DNA

| Oligo Name | Sequence |
|---|---|
| Human testis/sperm-specific | Forward: |
| histone H2B (TSH2B) | CAGACATCTCCTCGCATCAA (SEQ |
| | ID NO: 1) |
| | Reverse: |
| | GGAGGATGAAAGATGCGGTA (SEQ |
| | ID NO: 2) |
| Bromodomain Testis Associated | Forward: |
| (BRDT) | CCCTTTGGCCTTACCAACTT (SEQ ID |
| | NO: 3) |
| | Reverse: |
| | GCCCTCCCTTGAAGAAAAAC (SEQ |
| | ID NO: 4) |
| Zinc Finger CCCH-Type Containing 13 | Forward: TCTCGGTCCACTCGTGATG |
| (ZC3H13) | (SEQ ID NO: 5) |
| | Reverse: CCGGGATTCTTCTGGATATG |
| | (SEQ ID NO: 6) |
| Neighbor of BRCA1 gene 2 (NBR2) | Forward: |
| | TGTTATTTTTCGGGTTCAGCTT (SEQ |
| | ID NO: 7) |
| | Reverse: |
| | GATTGGCTCTTACCACTTGTCC (SEQ |
| | ID NO: 8) |
| Glyceraldehyde-3-Phosphate | Forward: |
| Dehydrogenase | -TCGACAGTCAGCCGCATCT (SEQ |
| Peptidyl-Cysteine S-Nitrosylase | ID NO: 9) |
| (GAPDH) | Reverse: CTAGCCTCCCGGGTTTCTCT |
| | (SEQ ID NO: 10) |

### Bioinformatics Analysis

Cutadapt was used to remove the adapter sequence from raw sequencing reads. Cleaned reads were aligned to the human GRCh38 reference genome using Burrows-Wheeler Aligner (BWA). MethylQA was used to generate genome density for Multiplexed MeDIP-Seq (Mx-MeDIP-Seq) data and reads mapping and CpG coverage statistics. Aligned reads were used to call peaks with MACS2 using input as a control to identify enriched areas in the genome based on performed methylated immunoprecipitation. Next, bdgcmp utility from the MACS2 package was used to deduct noise by comparing two signal tracks (IP and input) in bedGraph containing log 10 value of fold enrichment. To assess the similarity between individual and pooled samples first the percent of CG coverage of cleaned reads was compared. Next, the correlation of read counts on different regions for all different samples was performed. Before computing the correlation, first, all overlapping peaks regions between individual and pooled samples were merged into one bed file. The bed file contains all regions that should be considered for the coverage analysis. Then, multiBamSummary from deeptools computed the read coverages (number of unique reads mapped at a given nucleotide) over the merged bed file. The read coverage (number of unique reads mapped at a given nucleotide) over a large number of regions from each of the inputted BAM files (individual and pooled samples). Finally, deeptools plot correlation was used to visualize the multiBamSummary file (FIG. 2). Moreover, and through another method, the correlation between fold enrichment on CpG islands was calculated for the individual and pooled sample. ComputeMatrix from deeptools was used to calculate the log 10 value of fold enrichment scores of each base in the CpG islands. In this method, “gene body length” was divided by the “bin size” which was considered 50 in this study. The fold enrichment average in each bin across the CpG islands was then calculated. The common regions that overlap between two files were selected per genome region (CpG islands). Pearson correlation was employed to calculate the correlation between the average enrichment scores from the common region in two files (FIG. 2). Differential binding analysis and binding affinity of ChIP-seq peaksets were analyzed using R Bioconductor package DiffBind 2.17.0. Macs 2 used as a peak caller for chip-seq data and the peak files which contain intervals of a chromosome, a start and end position, and p-value and fold enrichment. Each peakset had an associated mapped sequencing reads (BAM) and a control read.

### Individual MeDIP and Mx-MeDIP Result in Similar Enrichment and Correlation

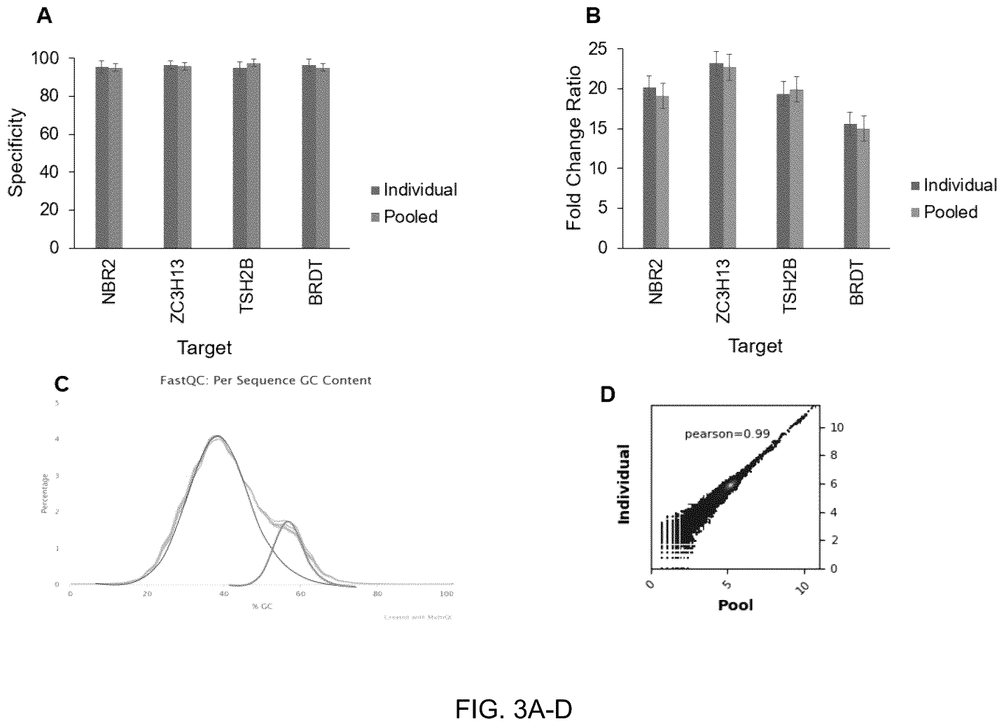

FIGS. 3A-3B show the results of enrichment analysis for individually processed immunoprecipitation and pooled samples. To do that, 10 different DNA libraries with unique adapters were carried through MeDIP. A pooled sample was also prepared to contain the same amount of each individual sample and simultaneously carried through Mx-MeDIP methodology. The final pooled product was amplified and cleaned up to remove any adaptor contamination. Fold enrichment for each sample was assessed with qPCR vs. input control to see if the pool results in a similar product compared to conventional individual MeDIP. As demonstrated in the plots of FIG. 3A, the average specificity was over 95% for all the assessed targets for both individual and pooled groups. In addition, similar specificity for each target was observed. Furthermore, there was no significant difference between individual and pooled samples, indicating that multiplexing can result in comparable specificity. The fold change ratio vs. input was also comparably similar for each target in both individual and pooled groups for all targets (NBR2, ZC3H13, TSH2B, BRDT).

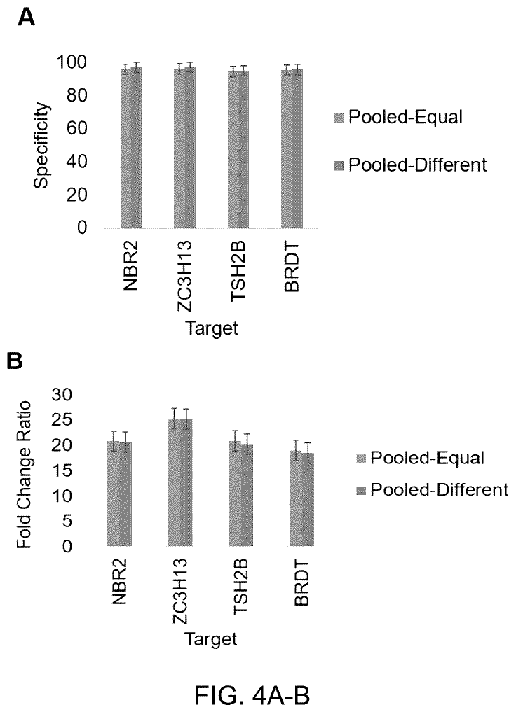

As shown in the FIG. 3B, fold change ratio value was between 15 for BRDT and 24 for ZC3H13. To further evaluate and compare individual vs. pooled samples, both groups were sequenced. First, basic quality control checks and % GC content calculations on obtained reads were carried out using FastQC. The sequencing of the methylated enriched DNA yielded approximately 55 million clean reads per sample. All samples performed well, with mean Phred scores above 35 along with the entire read. FIG. 3C shows empirical GC content in the MeDIP enriched samples, the there is a clear enrichment of GC content. Since both individual and pooled samples contain a portion of DNA that is enriched in GC content, the inventors expected to see a shifted distribution, as illustrated in FIG. 4C to the right side of the normal genomic DNA GC distribution. The shifted distribution was observed to be similar for both individual and pooled groups. The quality control analysis also revealed that both groups (individual and pooled) have similar % GC content (41%).

Next, the extent to which pooled and individual samples were reproducible and associated were determined using correlation scatter plots. To do that, Pearson correlation coefficients (R values) were computed using multiBamSummary from deeptools. This is a common technique to calculate the correlation of read counts (number of unique reads mapped at a given nucleotide) on different regions for all different samples. multiBamSummary computes the read coverage for genomic regions (merged bed files from individual and pooled samples, the output of macs) for both individual and pooled samples and was highly correlated, as shown in a representative image in FIG. 3D. Overall, the average correlation values or Pearson correlation coefficients (R values) between the read coverages for individual and pooled samples was measured to be 0.9±0.04. These highly correlated data show the similarity of BAM files and read coverage on peaks regions for both individual and pooled samples. For the next data assessment between individual and pooled samples, fold enrichment per CpG island was evaluated for both groups. Fold enrichment values for every bin in the gene body were calculated. Then, the Pearson correlation coefficient between scores for the two groups (individual vs pool) was calculated. For all the groups the result of ComputeMatrix over CpG island was highly correlated with each other (Pearson correlation coefficient>0.95). Therefore, each individual and pooled sample have peaks called on a similar region on CpG island. To evaluate the resolution of this method we prepared 200 ng DNA pools containing four different DNA samples. In one pool there was equal distribution (25%) of each sample. In other words, 50 ng of each sample was added to the pool to make 200 ng of starting material. Each sample had a different unique adapter. Another pool was prepared adding different amounts of each DNA sample. 20 ng of first DNA sample making 10% of the pool, 40 ng of the second sample making 20% of the pool, 60 and 80 ng of the third and fourth ones making 30% and 40% of the pool respectively. These two pools carried through multiplexing-MeDIP. This experiment was repeated three times. Specificity and fold enrichment were assessed using qPCR. Read depth for each sample in the pool was also analyzed after sequencing. To compare the MeDIP efficiency we first performed qPCR analysis. As demonstrated in FIGS. 4A-4B, specificity was more than 95% for all four positive targets and there was no difference observed between equally of differentially distributed pools (FIG. 4A). Also, the fold enrichment plot (FIG. 4B) showed that these two pools had similar fold enrichment ratios. These results suggested that, since each pool contained the same final amount of DNA (200 ng), the final enrichment and specificity were essentially the same, and were independent of the amount and proportion of each DNA sample in the starting pooled sample.

Multiplexing MeDIP has the advantages of being cost-effective, time-saving and requiring a small amount of starting DNA. The method of the current disclosure was tested on three replicates of different amounts of DNA from each source, 4 ng, 20 ng, 40 ng, 100 ng, 200 ng, and 400 ng. Each pool contains 4 different DNA samples with unique indexes which were pooled together. Precipitated DNA was analyzed using qPCR to determine the specificity and fold enrichment ratios. As expected, by increasing the starting material, the amount of overall DNA captured was increased. FIGS. 5A-5B show the specificity and fold enrichment plots for each pool. As FIG. 5A indicates, using as little as 10 ng of individual DNA sample in the method of the current disclosure results in acceptable specificity (>90%). However, there is a significant drop in sensitivity using 5 ng and 1 ng of individual DNA, resulting in 73 to 85% of specificity. Lowering the DNA amount to 1 ng reflecting the limitation in distinguishing methylated and unmethylated DNA however there is more than 70% chance to pool down methylated region. FIG. 5B illustrates the fold enrichment ratio achieved for starting the DNA amount as little as 1 ng in the pool. Using as little as 10 ng of DNA would result in acceptable specificity and fold enrichment ratio. However, there is a significant reduction in fold enrichment ratio of 1 ng and 5 ng of DNA compared to 50 ng and 100 ng of starting DNA. Moreover, there was no significant difference in specificity and fold enrichment ratio using 50 ng and 100 ng of starting DNA. The reduction of starting materials to 25 ng also results in comparable fold enrichment ratio and specificity vs 50 ng of DNA. However, similarly, the two experiments indicated that using as low as 5 ng and 1 ng would result in a significantly lower but still detectable fold enrichment ratio. Nevertheless, it is not recommended to use less than 10 ng of DNA for each individual sample. As long as the pool contains more than 10 ng of individual samples the specificity and fold enrichment ratios were acceptable.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for multiplexed detection of DNA methylation wherein the method comprises:
(a) contacting a micrococcal nuclease (MNase) to two or more individual samples comprising genomic DNA;
(b) purifying the MNase-contacted samples of step (a), whereby DNA samples comprising nucleosome-protected fragments are obtained;
(c) ligating sample-specific adapters to the nucleosome-protected fragments, wherein each of the two or more samples comprises a unique adapter and wherein adapter ligated DNA libraries are generated;
(d) pooling the two or more adapter-ligated DNA libraries to generate a pooled DNA library;
(e) performing methylated DNA immunoprecipitation on the pooled DNA library of step (d), thereby obtaining a pooled library enriched for methylated DNA;
(f) amplifying the DNA of step (e) using primers having specificity for the unique adaptors; and
(g) sequencing the amplified sample to detect DNA methylation.

2. The method of claim 1, further comprising analyzing sequences obtained in step (g) to identify, if present, aberrant DNA methylation in genomic DNA from the two or more sources.

3. The method of claim 1, wherein the pooled DNA library comprises genomic DNA from 2 to 15 individual sources.

4. The method of claim 1, wherein the pooled DNA library comprises at least 5 ng genomic DNA of each individual source.

5. The method of claim 1, wherein the pooled DNA library comprises at least 10 ng genomic DNA of each individual source.

6. The method of claim 1, wherein the pooled DNA library comprises less than 100 ng genomic DNA of each individual source.

7. The method of claim 1, wherein amplifying comprises quantitative polymerase chain reaction (qPCR).

8. The method of claim 1, wherein sequencing comprises next generation sequencing.

9. The method of claim 1, wherein the method has specificity greater than 70%.

10. The method of claim 9, wherein the method has specificity greater than 90%.

11. The method of claim 1, wherein the method can differentiate between 5-methylcytosine and 5-hydroxymethylcytosine.

12. The method of claim 1, wherein the micrococcal nuclease (MNase) reaction of step (a) is maintained at about 55° C.

13. The method of claim 1, wherein the micrococcal nuclease reaction of step (a) results in about 30% mono-nucleosomal DNA and about 25% di-nucleosomal DNA.

14. A kit for multiplexed detection of DNA methylation comprising:
(a) micrococcal nuclease; and
(b) an antibody specific for methylated DNA.

15. The kit of claim 14, further comprising sample-specific adapters and reagents for ligating the adapters to nucleosome-protected fragments.

16. The kit of claim 15, further comprising primers that hybridize with the sample-specific adapters.

17. The kit of claim 16, further comprising reagents for amplifying the nucleosome-protected fragments.

18. The kit of claim 17, wherein the reagents for amplifying comprise reagents for polymerase chain reaction (PCR).

19. The kit of claim 18, wherein the reagents for PCR comprise a high-fidelity polymerase.

20. The kit of claim 17, further comprising reagents for next generation sequencing.


SEQUENCE LISTING (10 sequences)

SEQ ID NO: 1 - DNA, 20 bp, Artificial Sequence
  Synthetic- Human testis/sperm-specific histone H2B (TSH2B) F
cagacatctcctcgcatcaa

SEQ ID NO: 2 - DNA, 20 bp, Artificial Sequence
  Synthetic- Human testis/sperm-specific histone H2B (TSH2B) R
ggaggatgaaagatgcggta

SEQ ID NO: 3 - DNA, 20 bp, Artificial Sequence
  Synthetic- Bromodomain Testis Associated (BRDT) F
ccctttggccttaccaactt

SEQ ID NO: 4 - DNA, 20 bp, Artificial Sequence
  Synthetic- Bromodomain Testis Associated (BRDT) R
gccctcccttgaagaaaaac

SEQ ID NO: 5 - DNA, 19 bp, Artificial Sequence
  Synthetic- Zinc Finger CCCH-Type Containing 13 (ZC3H13) F
tctcggtccactcgtgatg

SEQ ID NO: 6 - DNA, 20 bp, Artificial Sequence
  Synthetic- Zinc Finger CCCH-Type Containing 13 (ZC3H13) R
ccgggattcttctggatatg

SEQ ID NO: 7 - DNA, 22 bp, Artificial Sequence
  Synthetic- Neighbor of BRCA1 gene 2 (NBR2) F
tgttatttttcgggttcagctt

SEQ ID NO: 8 - DNA, 22 bp, Artificial Sequence
  Synthetic- Neighbor of BRCA1 gene 2 (NBR2)
gattggctcttaccacttgtcc

SEQ ID NO: 9 - DNA, 19 bp, Artificial Sequence
  Synthetic- Glyceraldehyde-3-Phosphate Dehydrogenase Peptidyl-Cysteine S-Nitrosylase (GAPDH) F
tcgacagtcagccgcatct

SEQ ID NO: 10 - DNA, 20 bp, Artificial Sequence
  Synthetic- Glyceraldehyde-3-Phosphate Dehydrogenase Peptidyl-Cysteine S-Nitrosylase (GAPDH) R
ctagcctcccgggtttctct